(19) United States
(12) Patent Application Publication
(10) Pub. No.: US 2025/0057735 A1
(43) Pub. Date: Feb. 20, 2025

(54) SOLID FRAGRANCING COMPOSITION

(71) Applicant: L'OREAL, Paris (FR)

(72) Inventors: Christine NOEL, CHEVILLY LARUE (FR); Véronique PERRAULT, CHEVILLY LARUE (FR)

(21) Appl. No.: 18/722,324

(22) PCT Filed: Dec. 21, 2022
(86) PCT No.: PCT/EP2022/087328

§ 371 Date: Jun. 20, 2024

(30) Foreign Application Priority Data
Dec. 22, 2021 (FR) FR2114241

(51) Int. Cl. A61K 8/02; A61K 8/37; A61K 8/67; A61K 8/92; A61Q 13/00

(52) U.S. Cl. CPC A61K 8/0229; A61K 8/375; A61K 8/67; A61K 8/922; A61K 8/927; A61Q 13/00

(57) ABSTRACT

The present invention relates to an anhydrous solid fragrancing composition, comprising:
i) at least 2% by weight, relative to the total weight of said composition, of one or more fragrancing substance(s);
ii) one or more natural waxes and/or waxes of natural origin; and
iii) one or more natural fatty substances and/or fatty substances of natural origin, other than said natural wax(es) and/or wax(es) of natural origin ii).

The invention also relates to a cosmetic process for fragrancing keratin materials, in particular the skin and notably the pulse points or the lips, comprising the application of such a solid fragrancing composition to said keratin materials.

### TECHNICAL FIELD

The present invention relates to the field of solid fragrances for fragrancing human keratin materials, and in particular the skin. The invention relates more particularly to a novel solid, anhydrous fragrancing cosmetic composition advantageously comprising compounds of natural origin.

The invention also relates to a cosmetic process for fragrancing keratin materials, in particular the skin and notably the pulse points, comprising the application of said solid fragrancing composition.

### PRIOR ART

In general, the formulation of environmentally friendly cosmetic products has become a major challenge for meeting new consumer expectations, in particular regarding natural and/or eco-friendly products, i.e. products whose design and development take into account their environmental impacts.

It is thus common practice to seek to replace synthetic compounds present in cosmetic compositions with natural ingredients and/or ingredients of natural origin.

These considerations have notably given rise to the development of green chemistry, also known as sustainable chemistry or ecological chemistry, which envisages the implementation of principles for reducing and eliminating the use or generation of environmentally unfriendly substances. The guiding principle of “Green Chemistry”, put forward in 1991, is to reduce or eliminate at source the use of hazardous substances in the design of new products.

These environmental problems concern all cosmetic products, notably fragrancing compositions.

The majority of fragrancing products are in the form of liquid, the fragrance of which is dissolved with alcohol (ethanol). In addition, there are on the market solid fragrances, also known as “perfume concretes”, without alcohol, presented in the form of dishes or sticks, which offer the consumer a new gesture for applying perfume. These solid fragrances have the advantage of being able to apply perfume to a precise spot, which is not always possible with a spray, and to avoid possible splashes of perfume.

Hitherto, the formulation of fragrancing compositions involved numerous products resulting from petrochemistry, for instance synthetic oils, polyalkylene waxes notably of C₁-C₆ such as polymethylene waxes, polyethylene waxes, or paraffin waxes, synthetic microcrystalline waxes, refined hydrocarbon waxes, etc.

In an effort to meet consumer expectations for natural products, “all natural” or “organic” solid fragrances have been proposed, and are promoted for their natural aspect.

Unfortunately, these solid fragrances are generally not up to consumers' expectations, in particular in terms of satisfactory transfer of the fragrancing composition onto the skin and the persistence of the fragrance, etc.

The reason for this is that consumers are in search of a product which is, admittedly, more natural, but without this being to the detriment of the desired properties, notably in terms of stability, staying power of the product, fragrancing and fragrance persistence, etc.

Proposing a solid fragrance with ingredients of natural origin is, however, a challenge given the many, occasionally contradictory, requirements that this type of formulation must meet. Indeed, it is imperative for these solid fragrances to combine both good properties in terms of hardness, so as to be able to keep a product in a good shape, for example in the form of a stick, but also good properties in terms of transfer onto the desired area, so as to allow the deposition of a sufficient amount of fragrancing composition to provide the desired odor.

In addition, these solid fragrances are put forward for their practical and portable aspect, allowing the consumer to apply them quickly and easily, and enabling the chosen areas to be fragranced in a simple application gesture. It is consequently essential for the application to be able to be done in an easy manner, without dribbling, and targeted on the desired areas, typically at the pulse points.

In the case of a perfume concrete packaged in the form of a dish, it must suffice to pass the finger over the perfume, without pressing too hard, and then to apply the perfume with the finger onto the desired areas. In the case of a solid fragrance in stick form, the stick must be sturdy enough so as not to crush or crumble upon application, while at the same time allowing a good transfer during application so as to allow the deposition of a sufficient amount of perfume.

Moreover, consumers expect a good persistence of the perfume once it has been applied, in other words an odor that persists over time, to avoid the need for repeated application actions. In other words, the fragrancing composition must be able to remain on the skin after application and must thus have satisfactory staying power, and the perfume must be detected even several hours after application.

The solid fragrancing composition according to the invention is precisely directed toward meeting these expectations.

### SUMMARY OF THE INVENTION

The present invention thus proposes the formulation of a novel solid fragrancing composition, which meets consumer expectations in terms of natural products, while at the same time satisfying the properties sought for this type of product, in particular making it possible to combine properties of good hardness, targeted and easy application to the desired areas of application and good staying power of the perfume.

More particularly, according to a first of its aspects, the invention relates to a solid fragrancing composition, in particular a cosmetic composition, said composition being anhydrous and comprising:
i) at least 2% by weight, relative to the total weight of said fragrancing composition, of one or more fragrancing substance(s);
ii) one or more natural waxes and/or waxes of natural origin; and
iii) one or more natural fatty substances and/or fatty substances of natural origin, other than said natural wax(es) and/or wax(es) of natural origin ii).

In the continuation of the specification, the fragrancing composition as defined according to the invention will be referred to more simply as the “solid fragrancing composition” or “solid fragrance”

The invention also relates to a cosmetic process for fragrancing keratin materials, comprising the application to said keratin materials of a solid fragrancing composition as defined above.

For the purposes of the present invention, the term “fragranced composition” or “fragrancing composition” is intended to denote any composition which leaves a perfume or odor on keratin materials after application.

A perfume is generally the result of a combination of different odorous substances which each provide a specific diffusing odor or “note” and which respectively evaporate at different periods. More precisely, each perfume has what is known as a “head note”, which is the odor that diffuses first when the perfume is applied or when the receptacle containing it is opened, a “heart or body note”, which corresponds to the full fragrance (given off for a few hours after the “head note”) and a “base note”, which is the most persistent odor (given off for several hours after the “body note”). The persistence of the base note corresponds to the remanence of the perfume.

For the purposes of the present invention, the term “keratin materials” preferably means human keratin materials, notably the skin, lips and/or keratin fibers, for example the eyelashes, in particular bodily and/or facial skin and the lips.

Preferably, the fragrancing composition according to the invention is intended for targeted application to the skin or the lips, in other words to specifically chosen areas of the skin or to the lips, and notably to the wrists, the hollows of the arms, behind the knees, the neck and the nape of the neck, and more specifically to the pulse points.

The term “pulse points” means the parts of the body where the heartbeat is felt, such as the temples, the areas of skin behind the ears, on the inside of the wrists, on the inside of the elbow, in the hollow of the neck, and at the base of the throat.

A composition according to the invention is suitable for application to keratin materials, notably to the skin and/or the lips, and thus generally comprises a physiologically acceptable medium, i.e. a medium that is compatible with the skin or the lips.

It is preferably a cosmetically acceptable medium, i.e. a medium which has a pleasant color, odor and feel and which does not cause any unacceptable discomfort, i.e. stinging, tautness or redness, liable to discourage the user from applying this composition.

In the context of a fragrancing cosmetic composition intended for application to the lips, the ingredients of the composition may also be edible.

Advantageously, the fragrancing composition according to the invention meets the many requirements sought for a solid fragrance, while at the same time using products of natural origin, in particular being predominantly, or even exclusively, formed from products of natural origin.

For the purposes of the present invention, the term “natural” is intended to characterize any compound obtained directly from the earth or the soil, or from plants or animals, via, where appropriate, one or more physical processes, for instance milling, refining, distillation, purification or filtration, or else from a biotechnological process, notably obtained from microbiological or cell cultures, for example from fungi or from bacteria. Among the “natural” dyes or pigments are compounds that are present in nature and that can be reproduced by chemical (semi) synthesis.

For the purposes of the present invention, the term “of natural origin” is intended to denote any compound obtained from a natural substance which has undergone one or more associated chemical or industrial treatments, giving rise to changes which do not affect the essential qualities of this substance. Mention may be made, as nonlimiting examples of additional chemical and industrial treatments bringing about modifications which do not affect the essential qualities of a natural compound, of those allowed by the controlling bodies, such as Ecocert (Reference system for biological and ecological cosmetic products, January 2003), or defined in recognized handbooks in the field, such as Cosmetics and Toiletries Magazine, 2005, Vol. 120, 9:10.

The term “biobased” means any component of plant or animal origin, preferably of plant origin.

In the rest of the text and for the purpose of simplification, the term “natural” which qualifies a compound designates both a natural compound and/or a compound of natural origin. According to the invention, a compound is considered to be natural when it is predominantly composed of natural constituents or constituents of natural origin, i.e. when the weight ratio of natural constituents or constituents of natural origin to non-natural constituents which make up the compound is greater than 1.

Preferably, the solid fragrancing compositions according to the invention are in the form of cast products, in a dish or jar, or in the form of wands or sticks. Advantageously, a solid fragrancing composition according to the invention is in the form of a stick.

A solid fragrancing composition according to the invention meets consumer expectations for “portable” products and small-format products (notably “pocket-size” products), which can be carried around everywhere and used in any place, since they can be applied quickly and easily.

A solid fragrancing composition according to the invention, whether or not it is malleable, combines particularly satisfactory resistance and rigidity properties, while at the same time leaving a good deposit on the keratin material, in particular on the skin.

Advantageously, a solid fragrancing composition according to the invention has particularly satisfactory hardness (or rigidity) and stability properties, making it possible to keep a product in good shape, in particular one which does not crush, crumble or collapse at the time of application.

A fragrancing composition according to the invention simultaneously makes it possible to give a sufficient deposit at the chosen application area, so as to deposit a sufficient amount of fragrancing composition to provide the desired odor.

Advantageously, the deposition of a sufficient amount of fragrancing composition is easy, and can be done in a simple application gesture. For example, a simple to and fro motion of the stick on the area to be fragranced suffices to deposit a sufficient amount of the composition. In the case of a solid fragrancing composition in the form of a dish, a simple passage of the finger or any application device, without pressing too hard, makes it possible to take up a sufficient amount of fragrancing composition, and the fragrancing composition is then easily transferred onto the chosen application area by a simple application gesture, for example by a single passage of the finger over the desired area.

Moreover, once applied to said keratin materials, in particular to the skin, the fragrancing composition according to the invention has good staying power. In particular, the deposit has good persistence of the fragrance; in other words, the fragrance persists over time, after application to the skin.

Advantageously, a solid fragrancing composition according to the invention can incorporate high levels of fragrancing substance(s), in particular as defined below, without affecting the other properties of the fragrancing composition, as mentioned previously. Advantageously, the solid fragrancing composition according to the invention may comprise more than 5% by weight of fragrancing substance(s), in particular at least 8% by weight, more particularly at least 9% by weight, even more particularly at least 10% by weight, or even 15% by weight and even up to 25% by weight, in particular up to 30% by weight of fragrancing substance(s), relative to the total weight of the fragrancing composition.

According to a particular embodiment, the solid fragrancing composition of the invention comprises at least 5% by weight of fragrancing substance(s), more particularly at least 8% by weight, in particular from 8% to 40% by weight, preferably from 9% to 30%, more preferentially from 10% to 25% by weight of fragrancing substance(s), relative to the total weight of the fragrancing composition.

According to a particular embodiment of the invention, the solid fragrancing composition comprises from 1% to 40% by weight of fragrancing substance(s), more particularly between 5% and 30% by weight, more preferentially between 10% and 25% by weight of fragrancing substance(s), relative to the total weight of the fragrancing composition.

The fragrancing composition may also advantageously, in addition to the abovementioned properties, act by forming an occlusive barrier on the surface of the keratin materials, for example on the lips, which helps to keep water on the lips, so as to improve their sensory properties (softness, etc.). In addition to the desired fragrancing properties, a solid fragrancing composition according to the invention can thus make it possible to preserve and/or stimulate over time the integrity of the lipid barrier of the stratum corneum of the skin, for example at the pulse points, and/or on the lips and to preserve and/or reinforce over time its natural barrier function.

Also, advantageously, the invention allows targeted application to the desired areas. In particular, the solid fragrancing composition can be applied to the specifically chosen areas, for example to the pulse points or the lips, without spreading or dribbling.

Other characteristics, variants and advantages of a solid fragrancing composition according to the invention, and of its implementation, will emerge more clearly on reading the following description and examples, which are given as nonlimiting illustrations of the invention.

In the continuation of the text, the expressions “between . . . and . . . ”, “ranging from . . . to . . . ” and “varying from . . . to . . . ” are equivalent and are intended to mean that the limits are included, unless otherwise indicated. The expression “at least one” is equivalent to “one or more”.

### DETAILED DESCRIPTION

As indicated previously, the fragrancing composition according to the invention is solid, anhydrous, and comprises:
i) at least 2% by weight, relative to the total weight of said fragrancing composition, of one or more fragrancing substance(s);
ii) one or more natural waxes and/or waxes of natural origin; and
iii) one or more natural fatty substance(s) and/or fatty substance(s) of natural origin, other than said natural wax(es) and/or wax(es) of natural origin ii), which are preferably liquid at 20° C. and at atmospheric pressure.

For the purposes of the present invention, the term “solid composition” means a composition which, at 20° C. and atmospheric pressure (760 mmHg), does not flow under its own weight, in other words is not liquid; the solid composition may or may not be malleable.

As indicated previously, a solid fragrancing composition according to the invention advantageously has good hardness properties. Advantageously, these good hardness properties ensure that the consumer can use the solid fragrance according to the invention without it being crushed, running or collapsing at the time of use.

In addition, it is of great interest that the solid composition can be applied precisely, without any diffusion effect being noticed on the surface of the composition in contact with the substrate, notably in contact with keratin materials, such as the skin. Notably, in the case of a solid fragrance in stick form according to the invention, said stick does not crush when the user places it directly in contact with the targeted areas. In the case of a solid fragrance in the form of a dish, the user can also pass his or her finger over the surface of the solid fragrance, without the perfume collapsing or being crushed in the dish.

The hardness of a solid fragrancing composition according to the invention can be assessed by measuring the shear hardness of the solid fragrancing composition formulated in the form of a stick.

The shear hardness of a stick can be measured by means of the “cheese wire” method at a temperature of 20° C., for example using a Ruptometer machine from Ametek.

The “cheese wire” method more particularly consists in transversely cutting a wand of said solid composition, which is preferably a circular cylinder, by means of a rigid tungsten wire 250 μm in diameter, by moving the wire relative to the stick at a speed of 100 mm/minute. The stick is stored at 20° C. for 24 hours before measuring the hardness.

The hardness value corresponds to the maximum shear force exerted by the wire on the stick at 20° C. The measurement is repeated three times and an average is taken. The average value is expressed in grams. This operation is performed on the longest length of the stick section when the stick is in the form of a cylinder with an elongated cross section.

According to a particular embodiment, a solid fragrancing composition according to the invention advantageously has a shear hardness, measured at 20° C. according to the protocol described previously, of greater than or equal to 100 g, in particular greater than or equal to 120 g, preferably greater than or equal to 130 g, in particular between 130 g and 300 g. Preferably, a solid fragrancing composition according to the invention in the form of a stick has a shear hardness, measured at 20° C. according to the protocol described previously, of greater than or equal to 150 g, in particular greater than or equal to 200 g, notably between 220 and 250 g.

Preferably, a solid fragrancing composition according to the invention in the form of a concrete (in a dish) has a shear hardness, measured according to the protocol described previously, at 20° C., of greater than or equal to 100 g, in particular between 120 g and 150 g, notably between 130 g and 140 g.

For the purposes of the present invention, the term “anhydrous” refers to a composition with a water content of less than 5% by weight, in particular less than 2% by weight, preferably less than 1% by weight, or even less than 0.5% by weight, relative to the total weight of the composition. Even more preferentially, a composition according to the invention is totally free of water. In particular, an anhydrous composition is a composition in which water is not deliberately added, but may be present in trace amount in the various compounds used in the composition.

### i) Fragrancing Substance(s)

A solid fragrancing composition according to the invention advantageously incorporates a significant content of fragrancing substance(s), without this affecting the properties of the composition.

The term “fragrancing substance”, also called an “odorous substance”, means any perfume, any odorous starting material or aroma that is capable of giving off a pleasant odor. For the purposes of the present invention, “fragrancing”, “odorous” or “odoriferous” substances are synonymous.

Advantageously, a solid fragrancing composition according to the invention comprises at least 2% by weight of fragrancing substance(s), advantageously at least 5% by weight, in particular at least 8% by weight, more particularly at least 9% by weight, even more particularly at least 10% by weight, even 15% by weight and even up to 25% by weight, in particular up to 30% by weight of fragrancing substance(s), relative to the total weight of the fragrancing composition.

According to a particular embodiment, the solid fragrancing composition of the invention comprises at least 5% by weight of fragrancing substance(s), more particularly at least 8% by weight, in particular from 8% to 40% by weight, preferably from 9% to 30%, more preferentially from 10 to 25% by weight of fragrancing substance(s), relative to the total weight of the fragrancing composition.

According to a particular embodiment of the invention, the solid fragrancing composition comprises from 1% to 40% by weight of fragrancing substance(s), more particularly between 5% and 30% by weight, more preferentially between 10% and 25% by weight of fragrancing substance(s), relative to the total weight of the fragrancing composition.

Fragrancing substances are compositions notably containing the starting materials described in S. Arctander, Perfume and Flavor Chemicals (Montclair, N.J., 1969), in S. Arctander, Perfume and Flavor Materials of Natural Origin (Elizabeth, N.J., 1960) and in Flavor and Fragrance Materials-1991, Allured Publishing Co., Wheaton, III.

A fragrancing composition according to the invention may notably comprise at least one fragrancing substance chosen from essential oils, perfumes and aromas of synthetic or natural origin, and mixtures thereof.

They may be natural products, such as essential oils, absolutes, resinoids, resins, concretes, and/or synthetic products, such as terpene or sesquiterpene hydrocarbons, alcohols, phenols, aldehydes, ketones, ethers, acids, esters, nitriles or peroxides, which may be saturated or unsaturated, and aliphatic or cyclic.

According to the definition given in the international standard ISO 9235 and adopted by the Commission of the European Pharmacopoeia, an essential oil is an odorous product, generally of complex composition, obtained from a botanically defined plant starting material, either by steam distillation, or by dry distillation, or by an appropriate mechanical process without heating (cold pressing). The essential oil is generally separated from the aqueous phase via a physical process which does not result in any significant change in the composition.

The choice of the method for obtaining essential oils depends mainly on the starting material: its original state and its characteristics, its intrinsic nature. The “essential oil/plant starting material” yield may be extremely variable depending on the plant: 15 ppm to more than 20%. This choice determines the characteristics of the essential oil, in particular viscosity, color, solubility, volatility, and richness or poorness in certain constituents.

Steam entrainment corresponds to vaporization, in the presence of steam, of a sparingly water-miscible substance. The raw material is placed in contact with boiling water or steam in an alambic. The steam entrains the essential oil vapor, which is condensed in the condenser and recovered as a liquid phase in a Florentine vase (or essence jar), where the essential oil is separated from the water by settling. The aqueous distillate that remains after the steam distillation, once the separation of the essential oil has been performed, is known as the “aromatic water” or “hydrolate” or “distilled floral water”.

Production by dry distillation consists in obtaining the essential oil by distillation of woods, barks or roots, without addition of water or steam, in a closed chamber designed so that the liquid is recovered at the bottom. Cade oil is the best known example of a product obtained in this way.

The method of production by cold pressing applies only to citrus fruits (Citrus spp.) via mechanical processes at room temperature. The principle of the method is as follows: the zests are torn into pieces and the contents of the secretory sacs that have been broken are recovered by a physical process. The conventional process consists in exerting an abrasive action on the entire surface of the fruit under a stream of water. After removal of the solid waste, the essential oil is separated from the aqueous phase by centrifugation. The majority of industrial installations actually allow simultaneous or sequential recovery of the fruit juices and of the essential oil. Essential oils are generally volatile and liquid at room temperature, which distinguishes them from “set” oils. They are more or less colored and their density is generally less than that of water. They have a high refractive index and most of them deflect polarized light. They are liposoluble and soluble in the usual organic solvents, distillable with steam, and very sparingly soluble in water.

Among the essential oils that may be used according to the invention, mention may be made of those obtained from plants belonging to the following botanical families: Abietaceae or Pinaceae, for example conifers; Amaryllidaceae; Anacardiaceae; Anonaceae, for example ylang ylang; Apiaceae, for example Umbelliferae, in particular dill, angelica, coriander, sca fennel, carrot or parsley; Araceae; Aristolochiaceae; Asteraceae, for example yarrow, artemisia, camomile, helichrysum; Betulaceae; Brassicaceae; Burseraceae, for example frankincense; Caryophyllaceae; Canellaceae; Cesalpiniaceae, for example copaifera (copaiba balsam); Chenopodaceae; Cistaceae, for example rock rose; Cyperaceae; Dipterocarpaceae; Ericaceae, for example gaultheria (wintergreen); Euphorbiaceac; Fabaccac; Geraniaceae, for example geranium; Guttiferae; Hamamelidaceae; Hernandiaceae; Hypericaceae, for example St-John's wort; Iridaceae; Juglandaceae; Lamiaceae, for example thyme, oregano, monarda, savory, basil, marjorams, mints, patchouli, lavenders, sages, catnip, rosemary, hyssop, balm, rosemary; Lauraceae, for example ravensara, sweet bay, rosewood, cinnamon, litsea; Liliaceae, for example garlic; Magnoliaceae, for example magnolia; Malvaceae; Meliaceae; Monimiaceae; Moraceae, for example hemp or hop; Myricaceae; Myristicaceae, for example nutmeg; Myrtaceae, for example eucalyptus, tea tree, paperbark tree, cajuput, backhousia, clove, myrtle; Oleaceae; Piperaceae, for example pepper; Pittosporaceae; Poaceae, for example citronella grass, lemongrass, vetiver; Polygonaceae; Renonculaceae; Rosaceae, for example roses; Rubiaceae; Rutaceae, for example all citrus plants; Salicaceae; Santalaceae, for example sandalwood; Saxifragaceac; Schisandraceae; Styracaceae, for example benzoin; Thymelaceae, for example agarwood; Tilliaceae; Valerianaceae, for example valerian, spikenard; Verbenaceae, for example lantana, verbena; Violaceae; Zingiberaceae, for example galangal, turmeric, cardamom, ginger; Zygophyllaceae.

Mention may also be made of the essential oils extracted from flowers (lily, lavender, rose, jasmine, ylang ylang, neroli), from stems and leaves (patchouli, geranium, petitgrain), from fruit (coriander, aniseed, cumin, juniper), from fruit peel (bergamot, lemon, orange), from roots (angelica, celery, cardamom, iris, sweet flag, ginger), from wood (pinewood, sandalwood, gaiac wood, rose of cedar, camphor), from grasses and gramineac (tarragon, rosemary, basil, lemongrass, sage, thyme), from needles and branches (spruce, fir, pine, dwarf pine) and from resins and balms (galbanum, elemi, benzoin, myrrh, olibanum, opopanax).

Examples of fragrancing substances are notably: geraniol, geranyl acetate, farnesol, borneol, bornyl acetate, linolool, linalyl acetate, linalyl propionate, linalyl butyrate, tetrahydrolinolool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, vetiveryl acetate, vetiverol, a-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranylacetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotropin, coumarin, eugenol, vanillin, diphenyl ether, citral, citronellal, hydroxycitronellal, damascone, ionones, methylionones, isomethylionones, solanone, irones, cis-3-hexenol and esters thereof, musk-indans, musk-tetralins, musk-isochromans, macrocyclic ketones, musk-macrolactones, aliphatic musks and ethylene brassylate, and mixtures thereof.

According to a preferred embodiment of the invention, a mixture of different fragrancing substances that generate in common a note that is pleasant to the user is used. Thus, according to a preferred embodiment, the fragrancing composition comprises a mixture of fragrancing substances, in particular at least two different fragrancing substances, and preferably at least three different fragrancing substances.

The fragrancing substances will preferably be chosen such that they produce notes (head, heart and base) in the following families: citrine, aromatic, floral notes, in particular pink flowers and white flowers, spicy, woody, gourmand, chypre, fougere, leathery, musk.

According to a particular embodiment, the fragrancing substance(s) used in a solid fragrancing composition according to the invention are different from essential oils.

Advantageously, a solid fragrancing composition according to the invention uses one or more natural fragrancing substances, preferably producing notes in the following families: floral notes, in particular pink flowers and white flowers, gourmands, and mixtures thereof.

In particular, the fragrancing substances i) according to the invention are distinct from the fatty substances considered according to the invention and detailed hereafter, in particular distinct from natural fatty substance(s) and fatty substance(s) of natural origin iii) and more particularly distinct from natural oil(s) and oil(s) of natural origin that may be introduced as the fatty substance(s) iii) as detailed hereafter.

### Natural Fatty Substances

As mentioned previously, a solid fragrancing composition according to the invention combines several natural fatty substances (which means natural fatty substances and/or fatty substances of natural origin), including ii) at least one natural wax.

The fatty substances may be fatty substances that are liquid at room temperature and atmospheric pressure, such as oils, or fatty substances that are solid at room temperature and atmospheric pressure, such as waxes, pasty fatty substances, butters, gums and mixtures thereof. For the purposes of the invention, the term “room temperature” means a temperature equal to 25° C.

According to a particular embodiment, a composition according to the invention comprises a mixture of one or more solid natural fatty substances, including ii) one or more natural waxes, and iii) one or more liquid natural fatty substances, in particular one or more natural oils.

In particular, the one or more natural fatty substances according to the invention are distinct from the fragrancing substances i) considered according to the invention and detailed hereabove.

### ii) Natural Wax(es)

As indicated previously, a solid fragrancing composition according to the invention comprises, as natural fatty substance, at least one natural wax (which means natural wax(es) and/or wax(es) of natural origin).

For the purposes of the present invention, the term “wax” generally means a lipophilic compound that is solid at room temperature (25° C.), with a solid/liquid reversible change of state, having a melting point of greater than or equal to 30° C., which may be up to 200° C. and notably up to 120° C.

The melting point of the wax can be measured using a differential scanning calorimeter (DSC), for example the calorimeter sold under the name DSC 30 by the company Mettler. Preferably, the measuring protocol is as follows:

A sample of 5 mg of wax placed in a crucible is subjected to a first temperature ramp passing from −20° C. to 100° C., at a heating rate of 10° C./minute, it is then cooled from 100° C. to −20° C. at a cooling rate of 10° C./minute and is finally subjected to a second temperature ramp passing from −20° C. to 100° C. at a heating rate of 5° C./minute. During the second temperature ramp, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The wax may notably have a hardness ranging from 0.05 MPa to 15 MPa and preferably ranging from 6 MPa to 15 MPa. The hardness is determined by measuring the compressive force, measured at 20° C. using the texture analyzer sold under the name TA-TX2i by the company Rheo, equipped with a stainless-steel cylinder with a diameter of 2 mm, travelling at a measuring speed of 0.1 mm/second, and penetrating the wax to a penetration depth of 0.3 mm.

The waxes that may be used in the solid fragrancing compositions according to the invention are advantageously natural. The term “natural” wax is intended to denote any wax which pre-exists in nature or which can be converted, extracted or purified from natural compounds which exist in nature.

Preferably, a solid fragrancing composition does not comprise waxes other than natural waxes.

The natural waxes of the invention may be chosen more particularly from waxes of animal, plant or mineral origin, and mixtures thereof. The waxes, for the purposes of the invention, may be those generally used in the cosmetic or dermatological fields.

The natural waxes may notably be polar or apolar hydrocarbon-based waxes, optionally including ester or hydroxyl functions.

The term “hydrocarbon-based wax” means a wax formed essentially from, or even consisting solely of, carbon and hydrogen atoms.

Preferably, a composition may comprise at least one natural polar wax.

For the purposes of the present invention, the term “polar wax” means a wax whose solubility parameter at 25° C., is other than 0 (J/cm³)^{1/2}. In particular, the term “polar wax” means a wax whose chemical structure is formed essentially from, or even constituted by, carbon and hydrogen atoms, and comprising at least one highly electronegative heteroatom.

The polar waxes may be hydrocarbon-based waxes, in particular ester waxes and/or alcohol waxes.

Preferably, a solid fragrancing composition according to the invention comprises one or more polar waxes of ester type.

According to the invention, the term “ester wax” means a wax comprising at least one ester function. The ester waxes may also be hydroxylated.

As ester waxes, use may notably be made of beeswax, carnauba wax, candelilla wax, lanolin wax, rice bran wax, ouricury wax, alfalfa wax, cork fiber wax, sugar cane wax, Japan wax, sumac wax, montan wax, orange wax, laurel wax, jojoba wax, sunflower wax, lemon wax, olive wax, berry wax; and also waxes obtained by catalytic hydrogenation of animal or plant oils containing linear or branched C₈-C₃₂ fatty chains, such as hydrogenated jojoba oil, hydrogenated sunflower oil, hydrogenated castor oil, hydrogenated coconut kernel oil, and also waxes obtained by hydrogenation of castor oil esterified with cetyl alcohol.

Preferably, said natural wax(es) ii) of the solid fragrancing composition according to the invention are chosen from hydrocarbon-based waxes, in particular ester waxes and more particularly chosen from carnauba wax, beeswax, notably polyglycerolated beeswax, candelilla wax, hydrogenated jojoba oil and mixtures thereof.

According to a particular embodiment, a solid fragrancing composition according to the invention comprises at least one natural ester wax, in particular chosen from carnauba wax, beeswax, notably polyglycerolated beeswax, candelilla wax, hydrogenated jojoba oil or a mixture of these waxes, preferably chosen from carnauba wax, candelilla wax, hydrogenated jojoba oil or a mixture of these waxes.

In a particular embodiment, a composition according to the invention does not comprise any wax other than said natural wax(es) of ester type, in particular as mentioned previously.

According to a particular embodiment, the content of natural wax(es), preferably of ester wax type, in particular as described previously, in a solid fragrancing composition according to the invention is between 5% and 50% by weight, in particular between 8% and 40% by weight, more particularly between 10% and 35% by weight, notably between 15% and 30% by weight, preferably between 20% and 30% by weight, relative to the total weight of said composition.

Preferably, the natural wax(es) of a fragrancing composition according to the invention are chosen from hydrocarbon-based waxes, in particular ester waxes and more particularly chosen from carnauba wax, beeswax, notably polyglycerolated beeswax, candelilla wax, hydrogenated jojoba oil and mixtures thereof, in particular from carnauba wax, candelilla wax, hydrogenated jojoba oil and mixtures thereof.

According to a particular embodiment, a solid fragrancing composition according to the invention comprises a mixture of natural waxes comprising, or even consisting of, waxes of ester type, in particular chosen from carnauba wax, beeswax, notably polyglycerolated wax, candelilla wax, hydrogenated jojoba oil and mixtures thereof, in particular from carnauba wax, candelilla wax, hydrogenated jojoba oil and mixtures thereof.

Preferably, a solid fragrancing composition comprises at least carnauba wax and/or beeswax, candelilla wax and hydrogenated jojoba oil.

Preferably, a solid fragrancing composition comprises at least carnauba wax, candelilla wax and hydrogenated jojoba oil.

In a particular embodiment, a solid fragrancing composition according to the invention comprises a mixture of natural waxes comprising, or even being formed from:
carnauba wax and/or beeswax, which is notably polyglycerolated, notably carnauba wax;
in particular in a content ranging from 1% to 15%, in particular from 2% to 10%; notably from 2% to 8% by weight and more particularly from 3% to 7% by weight, relative to the total weight of said fragrancing composition;
candelilla wax, in particular in a content ranging from 1% to 15% by weight, in particular from 2% to 12% by weight, more particularly from 4% to 12% by weight, notably from 5% to 10% by weight, relative to the total weight of said fragrancing composition; and
hydrogenated jojoba oil, in particular in a content ranging from 5% to 25% by weight, in particular from 8% to 20% by weight and more particularly from 10% to 15% by weight, relative to the total weight of said fragrancing composition.

In particular, the one or more natural waxes ii) according to the invention are distinct from the fragrancing substances i) considered according to the invention and detailed hereabove.

### iii) Natural Fatty Substance(s) other than the Waxes ii)

In addition to ii) one or more natural waxes, notably as defined previously, the solid fragrancing composition according to the invention comprises iii) one or more natural fatty substances, which are preferably liquid(s) at room temperature (20° C.) and at atmospheric pressure (760 mmHg); preferably, said natural fatty substance(s) iii) are chosen from volatile or nonvolatile natural oils (which means volatile or nonvolatile natural oils and/or volatile or nonvolatile oils of natural origin), and mixtures thereof. Said oils may be monounsaturated, polyunsaturated or saturated, linear or branched oils.

For the purposes of the invention, the term “volatile oil” refers to any oil that is capable of evaporating on contact with the skin in less than one hour, at room temperature and atmospheric pressure.

The term “nonvolatile oil” refers to an oil that remains on the skin at room temperature and atmospheric pressure for at least several hours, and that notably has a vapor pressure of less than 0.01 mmHg (1.33 Pa).

These volatile or nonvolatile oils may be, for example, hydrocarbon-based oils. The term “hydrocarbon-based oil” means an oil mainly containing hydrogen and carbon atoms and preferably consisting solely of carbon and hydrogen atoms. The natural oils may be chosen from mineral, plant and animal oils, preferably from mineral and plant oils, and more preferentially oils of plant origin.

In particular, the one or more natural oils according to the invention are distinct from the fragrancing substances i) considered according to the invention and detailed hereabove.

Perhydrosqualene may be mentioned as an oil of animal origin.

As oils of mineral origin, mention may be made of liquid paraffin and liquid petroleum jelly.

According to a preferred embodiment, a solid fragrancing composition according to the invention comprises at least, as natural fatty substance(s) iii), other than said natural wax(es), one or more volatile or nonvolatile natural oils, preferably of plant origin, in particular chosen from hydrocarbon-based oils of plant origin and more preferentially from ester oils of plant origin.

These natural oils of ester type are preferably nonvolatile.

Among the ester oils of plant origin, mention may be made of fatty acid triesters of glycerol (triglycerides) in which the fatty acids may have varied chain lengths from C₄ to C_{24,} these chains possibly being linear or branched, and monounsaturated, polyunsaturated or saturated.

These hydrocarbon-based plant oils may optionally be refined and deodorized.

These hydrocarbon-based plant oils with high triglyceride contents may notably be sweet almond oil, avocado oil, castor oil, olive oil, jojoba oil, sunflower oil, wheat germ oil, sesame oil, notably refined sesame oil, groundnut oil, grapeseed oil, soybean oil, rapeseed oil, safflower oil, coconut oil, coconut kernel oil, corn oil, hazelnut oil, shea olein, palm oil, apricot kernel oil or beauty-leaf oil.

Preferably, a solid fragrancing composition according to the invention comprises one or more oils of plant origin with a high triglyceride content, preferably chosen from castor oil, sesame oil, in particular refined sesame oil, caprylic/capric acid triglycerides (coconut oil, coconut kernel oil) and mixtures thereof.

According to a particular embodiment, the content of oil(s) of plant origin of triglyceride type, in a solid fragrancing composition according to the invention, is between 5% and 80% by weight, in particular between 10% and 80% by weight, notably between 20% and 75% by weight and more particularly between 30% and 70% by weight, notably between 40% and 65% by weight, relative to the total weight of said fragrancing composition.

As ester oils of plant origin, mention may also be made of fatty acid monoesters of plant origin, such as isopropyl myristate, myristyl myristate, isopropyl palmitate, cetyl palmitate, stearyl stearate and mixtures thereof.

Preferably, a solid fragrancing composition according to the invention comprises one or more fatty acid monoesters, also called oily fatty esters, of plant origin, preferably cetyl palmitate.

According to a particular embodiment, the content of fatty acid monoester(s), preferably cetyl palmitate, in a solid fragrancing composition according to the invention is between 0.1% and 15% by weight, in particular between 0.5% and 10% by weight, notably between 1% and 8% by weight and more particularly between 1% and 5% by weight, relative to the total weight of said fragrancing composition.

Thus, a fragrancing composition according to the invention may comprise, as natural fatty substance(s) iii), other than said natural wax(es), at least one oil of plant origin chosen from:
oils of plant origin with a high triglyceride content, preferably chosen from castor oil, sesame oil, in particular refined sesame oil, caprylic/capric acid triglycerides, and mixtures thereof;
fatty acid monoesters of plant origin, in particular chosen from isopropyl myristate, myristyl myristate, isopropyl palmitate, cetyl palmitate, stearyl stearate and mixtures thereof, preferably cetyl palmitate; and mixtures thereof.

According to a particular embodiment, a solid fragrancing composition according to the invention comprises, as natural fatty substance(s) iii), one or more natural oil(s), notably of plant origin, chosen in particular from nonvolatile hydrocarbon-based oils, preferably from ester oils and more preferentially chosen from oils of triglyceride type and/or fatty acid monoester as described previously, for example chosen from castor oil, sesame oil, in particular refined sesame oil, caprylic/capric acid triglycerides, cetyl palmitate and mixtures thereof.

In a particular embodiment, a composition according to the invention does not comprise any auxiliary oil, other than the natural oil(s) iii), in particular the oil(s) of plant origin, as described previously.

According to a particular embodiment, a solid fragrancing composition according to the invention comprises a total content of natural oil(s), preferably ester oil(s) of plant origin, ranging from 20% to 80%, in particular from 30% to 75% and more particularly from 40% to 70%, relative to the total weight of said composition.

Preferably, a solid fragrancing composition according to the invention comprises as ingredient iii) a mixture of:
one or more oils of plant origin of triglyceride type, for example caprylic/capric acid triglycerides, notably in a content ranging from 5% to 80% by weight, in particular between 10% and 80% by weight, notably between 20% and 75% by weight and more particularly between 30% and 70% by weight, notably between 40% and 65% by weight, relative to the total weight of said composition; and
one or more oily fatty monoesters of plant origin, in particular cetyl palmitate, preferably in a content of between 0.1% and 15% by weight, in particular between 0.5% and 10% by weight, notably between 1% and 8% by weight and more particularly between 1% and 5% by weight, relative to the total weight of said composition.

According to a particular embodiment, a solid fragrancing composition according to the invention comprises:
one or more natural waxes ii), in particular a mixture of natural waxes as previously defined, and
as natural fatty substance(s) iii), other than said natural wax(es), one or more natural oils, preferably a mixture of ester oils of plant origin, as defined previously, in a wax(es) ii)/oil(s) iii) weight ratio strictly less than 1, in particular less than or equal to 0.5, preferably between 0.2 and 0.8, in particular between 0.3 and 0.7.

A solid fragrancing composition according to the invention may also comprise at least one solid natural fatty substance other than the natural waxes ii), in particular chosen from pasty fatty substances, for example butters, gums, and mixtures thereof.

For the purposes of the present invention, the term “pasty compound” or “pasty fatty compound” means a lipophilic fatty compound which undergoes a reversible solid/liquid change of state, having anisotropic crystal organization in the solid state, and which comprises, at a temperature of 23° C., a liquid fraction and a solid fraction.

In other words, the starting melting point of the pasty compound may be less than 23° C. The liquid fraction of the pasty compound measured at 23° C. may represent 9% to 97% by weight of the compound. This fraction that is liquid at 23° C. preferably represents between 15% and 85% and more preferably between 40% and 85% by weight.

For the purposes of the invention, the melting point corresponds to the temperature of the most endothermic peak observed on thermal analysis (DSC) as described in the standard ISO 11357-3; 1999. The melting point of a pasty substance or of a wax may be measured using a differential scanning calorimeter (DSC), for example the calorimeter sold under the name MDSC 2920 by the company TA Instruments.

The measuring protocol may be that described previously for determining the melting point of the waxes.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound in order to pass from the solid state to the liquid state. The pasty compound is said to be in the solid state when all of its mass is in crystalline solid form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5° C. or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., constituted of a liquid fraction and a solid fraction.

The liquid fraction of the pasty compound measured at 32° C. preferably represents from 30% to 100% by weight of the compound, preferably from 50% to 100% and more preferably from 60% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same way as the heat of fusion consumed at 23° C. The pasty fatty substances may be synthetic compounds or compounds of natural origin. A pasty fatty substance may preferably be obtained by synthesis from starting materials of plant origin.

Advantageously, the pasty fatty substance is of natural origin, notably of plant origin. It may be chosen, for example, from natural butters.

As natural butters, mention may notably be made of butters of plant origin, such as mango butter, shea butter, cupuaçu butter, murumuru butter, cocoa butter, babassu butter, avocado butter, kokum butter, coconut butter, apricot kernel butter, Shorea robusta butter and urucum butter.

A s examples of natural gums, mention may be made of xanthan gum, guar gum, gum arabic, locust bean gum, acacia gum, carrageenans, cellulose gums, and mixtures thereof.

According to a particular embodiment, a solid fragrancing composition according to the invention comprises at least 40% by weight, in particular at least 50% by weight, notably at least 60% by weight, of natural fatty substances including at least one or more natural wax(es) ii).

In particular, the total content of natural fatty substances, including at least one or more natural wax(es) ii), in a solid fragrancing composition according to the invention may be between 40% and 98% by weight, in particular between 50% and 90% by weight, and more particularly between 59% and 80% by weight, relative to the total weight of said fragrancing composition.

According to a particular embodiment, a solid fragrancing composition combines with one or more natural waxes, in particular with a mixture of natural waxes, as described previously, one or more natural oils, in particular as described previously.

According to a particular embodiment, a solid fragrancing composition according to the invention comprises at least:
i) one or more fragrancing substance(s), preferably of natural origin, in particular as described previously;
ii) one or more natural waxes and/or waxes of natural origin, preferably chosen from hydrocarbon-based waxes, in particular of ester type and more preferentially from carnauba wax, beeswax, candelilla wax, hydrogenated jojoba oil and mixtures thereof; in particular from carnauba wax, candelilla wax, hydrogenated jojoba oil, and mixtures thereof; and
iii) one or more natural oils and/or oils of natural origin, preferably of plant origin and more preferentially chosen from ester oils as described previously, in particular chosen from oils of triglyceride type, fatty acid monoesters and mixtures thereof, for example chosen from castor oil, sesame oil, caprylic/capric acid triglycerides, cetyl palmitate and mixtures thereof.

According to a particular embodiment, a solid fragrancing composition according to the invention at least comprises:
at least 2% by weight, preferably at least 5% by weight, in particular at least 8% by weight, notably from 8% to 40% by weight, preferably from 9% to 30%, more preferentially from 10 to 25% by weight of (i) fragrancing substance(s), relative to the total weight of the composition; preferably chosen from fragrancing substance(s) of natural origin as described previously;
from 5% to 50% by weight, in particular from 8% to 40% by weight, more particularly from 10% to 35% by weight, notably from 15% to 30% by weight, preferably from 20% to 30% by weight, relative to the total weight of said composition, of ii) natural wax(es) and/or wax(es) of natural origin preferably chosen from waxes of ester type and more preferentially from carnauba wax, beeswax, candelilla wax, hydrogenated jojoba oil and mixtures thereof, in particular from carnauba wax, candelilla wax, hydrogenated jojoba oil and mixtures thereof; and
from 20% to 80%, in particular from 30% to 75% and more particularly from 40% to 70%, relative to the total weight of said composition, of iii) natural oil(s) and/or oil(s) of natural origin, preferably of plant origin and more preferentially chosen from ester oils,
in particular chosen from oils of triglyceride type and/or fatty acid monoesters as described previously, for example chosen from castor oil, sesame oil, in particular refined sesame oil, caprylic/capric acid triglycerides, cetyl palmitate and mixtures thereof.

In a particular embodiment, a solid fragrancing composition according to the invention at least comprises:
one or more fragrancing substances, preferably of natural origin, and
carnauba wax, and/or
beeswax, in particular polyglycerolated beeswax, and/or
candelilla wax, and
hydrogenated jojoba oil, and/or
cetyl palmitate, and/or
triglycerides.

In a more particular embodiment, a solid fragrancing composition according to the invention at least comprises:
one or more fragrancing substances, preferably of natural origin, and
carnauba wax, and/or
beeswax, in particular polyglycerolated beeswax, and/or
candelilla wax, and
hydrogenated jojoba oil, and
cetyl palmitate, and/or
triglycerides.

In an even more particular embodiment, a solid fragrancing composition according to the invention at least comprises:
one or more fragrancing substances, preferably of natural origin,
carnauba wax and/or beeswax, which is notably polyglycerolated,
candelilla wax,
hydrogenated jojoba oil,
cetyl palmitate, and
triglycerides.

In another even more particular embodiment, a solid fragrancing composition according to the invention comprises at least:
one or more fragrancing substances, preferably of natural origin,
carnauba wax,
candelilla wax,
hydrogenated jojoba oil,
cetyl palmitate, and
triglycerides.

Even more particularly, a solid fragrancing composition according to the invention may comprise at least:
at least 2% by weight, preferably at least 5% by weight, in particular at least 8% by weight, notably from 8% to 40% by weight, preferably from 9% to 30%, more preferentially from 10 to 25% by weight of fragrancing substance(s) i); preferably chosen from fragrancing substance(s) of natural origin as described previously;
from 1% to 15%, in particular from 2% to 10%; notably from 2% to 8% by weight and more particularly from 3% to 7% by weight, of carnauba wax and/or beeswax, which is notably polyglycerolated, in particular carnauba wax,
from 1% to 15% by weight, in particular from 2% to 12% by weight, more particularly from 4% to 12% by weight, notably from 5% to 10% by weight of candelilla wax,
from 5% to 25% by weight, in particular from 8% to 20% by weight and more particularly from 10% to 15% by weight of hydrogenated jojoba oil,
from 0.1% to 15% by weight, in particular from 0.5% to 10% by weight, notably 1% to 8% by weight and more particularly from 1% to 5% by weight of cetyl palmitate,
from 5% to 80% by weight, in particular from 10% to 80% by weight, notably from 20% to 75% by weight and more particularly from 30% to 70% by weight, notably from 40% to 65% by weight of triglycerides, the percentages being given relative to the total weight of said solid fragrancing composition.

### Other Compounds

A fragrancing composition according to the invention may also comprise any additional component usually used in cosmetics, such as fillers, dyestuffs, cosmetic active agents, for example preserving agents, antioxidants, UV-screening agents and emollients.

The additives are generally present in an amount for each of them of between 0.01% and 10% by weight relative to the total weight of the composition. Needless to say, a person skilled in the art will take care to select the constituents of the composition such that the properties of the solid fragrancing composition according to the invention are not, or are not substantially, adversely affected.

Advantageously, said additive(s) used in a solid fragrancing composition according to the invention are of natural origin, in particular of plant origin.

According to a particular embodiment, a solid fragrancing composition according to the invention also comprises at least one cosmetic active agent, preferably at least one hydrophilic cosmetic active agent.

As examples of cosmetic active agents, mention may be made of moisturizers, antioxidant compounds, sunscreens, humectants, mattifying agents, cicatrizing agents, antibacterial agents, vitamins and derivatives thereof, and mixtures thereof.

The active agent(s) may notably be chosen from:
vitamins and derivatives thereof, notably esters thereof;
humectants, such as urea, hydroxyureas, glycerol, polyglycerols, glyceryl glucoside, diglyceryl glucoside, polyglyceryl glucosides and xylityl glucoside;
antioxidant compounds, for example tocopherol (vitamin E), dibutyl pentaerythrityl tetrahydroxycinnamate;
antiaging active agents, such as hyaluronic acid compounds and salicylic acid compounds;
sunscreens; and
mixtures thereof.

In particular, a solid fragrancing composition according to the invention may comprise at least one antioxidant compound, notably chosen from tocopherol (vitamin E), dibutyl pentaerythrityl tetrahydroxycinnamate and mixtures thereof.

Such active agents may be present in a composition according to the invention in a content ranging from 0.01% to 5.0% by weight and preferably from 0.05% to 2% by weight relative to the total weight of the composition.

According to a particular embodiment, a solid fragrancing composition according to the invention also comprises at least one filler of organic or mineral nature.

The term “filler” should be understood as meaning colorless or white solid particles of any shape which are provided in an insoluble form and dispersed in the medium of the composition. These fillers, of mineral or organic nature, give body or rigidity to the composition and/or softness and uniformity to the application. They are different from dyestuffs.

Among the fillers that may be used in the compositions according to the invention, mention may be made, for example, of silica, kaolin, bentone, starch, lauroyllysine, and fumed silica particles, which have optionally undergone a hydrophilic or hydrophobic treatment, and mixtures thereof.

The said filler(s) may be present in a composition according to the invention in a content ranging from 0.5% to 15% by weight and preferably from 1% to 10% by weight relative to the total weight of the composition.

According to a particular embodiment of the invention, a solid fragrancing composition contains at least one pigment, in particular chosen from natural pigments.

According to a particular embodiment of the invention, a solid fragrancing composition contains at least one nacre.

The term “nacres” should be understood as meaning colored particles of any form, which may or may not be iridescent, notably produced by certain molluscs in their shell, or alternatively synthesized, and which have a color effect via optical interference.

The nacreous pigments may be chosen, for example, from titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs. Examples of nacres that may also be mentioned include natural mica covered with titanium oxide, with iron oxide, with natural pigment or with bismuth oxychloride.

According to a particular embodiment, a solid fragrancing composition according to the invention may comprise:
at least 2% by weight, preferably at least 5% by weight, in particular at least 8% by weight, notably from 8% to 40% by weight, preferably from 9% to 30%, more preferentially from 10 to 25% by weight of fragrancing substance(s) i), relative to the total weight of the composition; preferably chosen from fragrancing substance(s) of natural origin;
carnauba wax and/or beeswax, which is notably polyglycerolated; in particular in a content ranging from 1% to 15%, in particular from 2% to 10%; notably from 2% to 8% by weight and more particularly from 3% to 7% by weight;
candelilla wax, in particular in a content ranging from 1% to 15% by weight, in particular from 2% to 12% by weight, more particularly from 4% to 12% by weight, notably from 5% to 10% by weight;
hydrogenated jojoba oil, in particular in a content ranging from 5% to 25% by weight, in particular from 8% to 20% by weight and more particularly from 10% to 15% by weight;
cetyl palmitate, in particular in a content ranging from 0.1% to 15% by weight, in particular from 0.5% to 10% by weight, notably from 1% to 8% by weight and more particularly from 1% to 5% by weight;
triglycerides, in particular in a content ranging from 5% to 80% by weight, in particular from 10% to 80% by weight, notably from 20% to 75% by weight and more particularly from 30% to 70% by weight, notably from 40% to 65% by weight; and
optionally one or more additives, in particular chosen from fillers, dyestuffs, cosmetic active agents, for example preserving agents, antioxidants, UV-screening agents and emollients; preferably one or more antioxidant compounds, for example chosen from tocopherol and/or dibutyl pentaerythrityl tetrahydroxycinnamate; preferably in a content ranging from 0.01% to 5.0% by weight, preferably from 0.05% to 2% by weight;
the percentages being given relative to the total weight of said solid fragrancing composition.

Advantageously, a solid fragrancing composition according to the invention in particular comprises less than 5% by weight, in particular less than 3% by weight, notably less than 2% by weight, preferably less than 1% by weight of alcohol(s), relative to the total weight of said composition, or is even completely alcohol-free.

Advantageously, a solid fragrancing composition according to the invention may comprise less than 5% by weight, in particular less than 3% by weight, notably less than 2% by weight, preferably less than 1% by weight of preserving agent(s) relative to the total weight of said composition, or even is completely free of preserving agent(s).

According to a particularly advantageous embodiment, a solid fragrancing composition according to the invention may be formed predominantly, or even totally, from green components, in particular of natural origin.

In particular, according to one embodiment variant, a solid fragrancing composition may be formed to more than 50% by weight, in particular to more than 70% by weight, notably to more than 80% or even to more than 90% by weight, or even exclusively formed, from natural components, in particular biobased components.

A solid fragrancing composition according to the invention may be manufactured via processes known to those skilled in the art, generally used in the cosmetic field. It may be manufactured, for example, by obtaining a homogeneous mixture of the various ingredients of the composition, followed by pouring the mixture obtained into a suitable mold such as a stick mold, or directly into the packaging articles (notably a case, jar or dish).

As indicated previously, advantageously, a solid fragrancing composition is packaged in the form of a “nomadic” and small-sized, easily transportable device, preferably in a pocket format.

A solid fragrancing composition according to the invention may be in the form of a wand or stick or may be poured into a dish or jar.

According to a particular embodiment, a solid fragrancing composition is in the form of a stick.

According to another particular embodiment, a solid fragrancing composition is in the form of a concrete packaged in a dish.

According to a particular embodiment, a solid fragrancing composition may advantageously be intended for application to the skin, in particular bodily or facial skin. Preferably, it is intended for application to one or more specific localized areas, for example on pulse points, such as the temples, the areas of skin behind the ears, on the inside of the wrists, on the inside of the elbow, in the hollow of the neck, and at the base of the throat.

The invention applies not only to fragrancing products whose main function is to perfume, but also to products for caring for, treating and/or making up the skin and the lips for which it is desired to simultaneously provide a pleasant odor. The composition according to the invention may thus constitute a fragrancing, care, treatment and/or makeup composition for keratin materials, and may notably be in the form of a stick. It may constitute, for example, products in stick form for antisun protection of facial skin, products for making up the skin, both of the human face and body, and notably in the form of colored makeup products such as foundations cast in stick or dish form, blushers and face powders, lipsticks, concealer products and temporary tattoo products, eye, eyebrow and/or hair makeup products such as eyeliners in pencil form and mascara cakes, and body hygiene products.

According to a particular embodiment, a solid fragrancing composition may be intended for application to the lips. It may be, for example, in the form of a lipstick or lip balm advantageously allowing, in addition to its intended use for making up and/or caring for the lips, to give the lips a pleasant odor.

As indicated previously, the present invention also relates to a cosmetic method for fragrancing keratin materials, in particular the skin, and notably the pulse points or the lips, comprising the application to the keratin materials of a solid fragrancing composition according to the invention.

The mode of application is notably adjusted to the form in which the solid fragrancing composition is presented.

According to a first variant, said step of application to the keratin materials is performed by placing the solid composition, notably packaged in the form of a stick, directly in contact with the desired area of application. The placing in contact may consist of a simple gesture of passing the stick over the desired area, for example to and fro.

According to another variant, said step of application to the keratin materials is performed by taking up, for example by finger, the solid fragrancing composition packaged in the form of a dish, for example by simply passing the finger over the solid composition, and then applying it to the desired area, for example by simply passing the finger over the desired area, for example to and fro.

The invention is illustrated in greater detail by the examples presented below. Unless otherwise indicated, the amounts indicated are expressed as mass percentages.

### EXAMPLES

In the examples that follow, the shear hardness is measured on solid compositions formulated in the form of a stick, by means of the “cheese wire” method, at a temperature of 20° C., using a Ruptometer machine from Ametek, according to the protocol described previously.

The hardness value thus corresponds to the maximum shear force exerted by the wire, measured on the stick at 20° C. The shear hardness is thus expressed in grams.

### Example 1

A solid fragrancing composition in stick form according to the invention was prepared as described below. The ingredients and the proportions thereof are indicated in Table 1 below. The amounts are given in g per 100 g of final composition.

TABLE 1

| | | | Fragrancing |
|---|---|---|---|
| | | Commercial | composition |
| Compounds | INCI name | reference | (g) |
| Carnauba wax | Copernica cerifera | Carnauba Wax | 4 |
| | (carnauba) wax/Copernica | 1 Flakes NF | |
| | cerifera cera | SP 63 | |
| Candelilla wax | Euphorbia cerifera | 7820 Light | 8 |
| | (candelilla) wax/Candelilla | Special | |
| | cera | Candelilla | |
| | | Real | |
| Hydrogenated jojoba | Hydrogenated jojoba oil | Jojoba Wax | 11 |
| oil | | Flakes | |
| Caprylic/capric acid | Caprylic/capric triglyceride | Miglyol 812 N | 33 |
| triglycerides | | | |
| Cetyl palmitate | Cetyl palmitate | Cutina CP | 3 |
| Castor oil | Ricinus communis (castor) | Crystal O | 15 |
| | seed oil/Ricinus communis | | |
| | seed oil | | |
| Refined sesame oil | Sesamum indicum (sesame) | Sesame Seed | 15 |
| | seed oil/Sesamum indicum | Oil Type C | |
| | seed oil | | |
| Dibutyl pentaerythrityl | Pentaerythrityl tetra-di-t- | Tinogard TT | 0.5 |
| tetrahydroxycinnamate | butyl | | |
| | hydroxyhydrocinnamate | | |
| Tocopherol | Tocopherol | Novatol 5-87 | 0.5 |
| | | 410218 | |
| Fragrancing substance | Perfume | | 10 |

All of the ingredients, except for the perfume, are mixed together and melted at 98° C., the perfume being introduced last so as not to deteriorate it, and the formulation is then poured into a mold at 40° C.

The shear hardness of this solid composition of Example 1 is 245 g.

It was found that, during the application of the composition in stick form, by exerting a normal force i.e. a force identical to the force exerted when applying a lipstick to lips, the wand does not crumble on the substrate.

The odor obtained after applying said solid composition of Example 1 to the substrate is very much the same as that of the fragrancing substance alone. The solid composition of Example 1 notably in the form of a stick allowed an easy and circumscribed application to the place where the solid composition of the invention was applied, contrary to the application of the fragrancing substance alone applied directly to the substrate which is difficult to control on the precise place to be fragranced. Moreover, it was found that the odor, after application of the solid composition of the invention, on the skin remains significantly more intense than that of the fragrancing substance alone, even after 4 hours.

### Example 2

A solid fragrancing composition in stick form according to the invention was prepared as described below. The ingredients and the proportions thereof are indicated in Table 2 below.

TABLE 2

| | | | Fragrancing |
|---|---|---|---|
| | | Commercial | composition |
| Compounds | INCI name | reference | (g) |
| Carnauba wax | Copernica cerifera | Carnauba Wax | 4 |
| | (carnauba) wax/Copernica | 1 Flakes NF | |
| | cerifera cera | SP 63 | |
| Candelilla wax | Euphorbia cerifera | 7820 Light | 8 |
| | (candelilla) wax/Candelilla | Special | |
| | cera | Candelilla | |
| | | Real | |
| Hydrogenated jojoba | Hydrogenated jojoba oil | Jojoba Wax | 11 |
| oil | | Flakes | |
| Caprylic/capric acid | Caprylic/capric triglyceride | Miglyol 812 N | 28 |
| triglycerides | | | |
| Cetyl palmitate | Cetyl palmitate | Cutina CP | 3 |
| Castor oil | Ricinus communis (castor) | Crystal O | 15 |
| | seed oil/Ricinus communis | | |
| | seed oil | | |
| Refined sesame oil | Sesamum indicum (sesame) | Sesame Seed | 15 |
| | seed oil/Sesamum indicum | Oil Type C | |
| | seed oil | | |
| Dibutyl pentaerythrityl | Pentaerythrityl tetra-di-t- | Tinogard TT | 0.5 |
| tetrahydroxycinnamate | butyl | | |
| | hydroxyhydrocinnamate | | |
| Tocopherol | Tocopherol | Novatol 5-87 | 0.5 |
| | | 410218 | |
| Fragrancing substance | Perfume | | 15 |

All of the ingredients, except for the perfume, are mixed together and melted at 98° C., the perfume being introduced last so as not to deteriorate it, and the formulation is then poured into a mold at 40° C.

The shear hardness of this solid composition of Example 2 is 250 g.

It was found that, during the application of the composition in stick form, by exerting a normal force i.e. a force identical to the force exerted when applying a lipstick to lips, the wand does not crumble on the substrate.

The odor obtained after applying the solid composition of Example 2 to the substrate is very much the same as that of the fragrancing substance alone. The solid composition of Example 2 notably in the form of a stick allowed an easy and circumscribed application to the place where the solid composition of the invention was applied, contrary to the application of the fragrancing substance alone applied directly to the substrate which is difficult to control on the precise place to be fragranced. Moreover, it was found that the odor, after application of the solid composition of the invention, on the skin remains significantly more intense than that of the fragrancing substance alone, even after 4 hours.

### Example 3

A solid fragrancing composition in stick form according to the invention was prepared as described below. The ingredients and the proportions thereof are indicated in Table 3 below.

TABLE 3

| | | | Fragrancing |
|---|---|---|---|
| | | Commercial | composition |
| Compounds | INCI name | reference | (g) |
| Carnauba wax | Copernica cerifera | Carnauba Wax | 5 |
| | (carnauba) wax/Copernica | 1 Flakes NF | |
| | cerifera cera | SP 63 | |
| Candelilla wax | Euphorbia cerifera | 7820 Light | 8 |
| | (candelilla) wax/Candelilla | Special | |
| | cera | Candelilla | |
| | | Real | |
| Hydrogenated jojoba | Hydrogenated jojoba oil | Jojoba Wax | 12 |
| oil | | Flakes | |
| Caprylic/capric acid | Caprylic/capric triglyceride | Miglyol 812 N | 26 |
| triglycerides | | | |
| Cetyl palmitate | Cetyl palmitate | Cutina CP | 3 |
| Castor oil | Ricinus communis (castor) | Crystal O | 15 |
| | seed oil/Ricinus communis | | |
| | seed oil | | |
| Refined sesame oil | Sesamum indicum (sesame) | Sesame Seed | 15 |
| | seed oil/Sesamum indicum | Oil Type C | |
| | seed oil | | |
| Dibutyl pentaerythrityl | Pentaerythrityl tetra-di-t- | Tinogard TT | 0.5 |
| tetrahydroxycinnamate | butyl | | |
| | hydroxyhydrocinnamate | | |
| Tocopherol | Tocopherol | Novatol 5-87 | 0.5 |
| | | 410218 | |
| Fragrancing substance | Pink floral fragrance | | 15 |

All of the ingredients, except for the perfume, are mixed together and melted at 98° C., the perfume being introduced last so as not to deteriorate it, and the formulation is then poured into a mold at 40° C.

The shear hardness of this solid composition of Example 3 is 230 g.

It was found that, during the application of the composition in stick form, by exerting a normal force i.e. a force identical to the force exerted when applying a lipstick to lips, the wand does not crumble on the substrate.

The odor obtained after applying the solid composition of Example 3 to the substrate is very much the same as that of the fragrancing substance alone. The solid composition of Example 3 notably in the form of a stick allowed an easy and circumscribed application to the place where the solid composition of the invention was applied, contrary to the application of the fragrancing substance alone applied directly to the substrate which is difficult to control on the precise place to be fragranced. Moreover, it was found that the odor, after application of the solid composition of the invention, on the skin remains significantly more intense than that of the fragrancing substance alone, even after 4 hours.

### Example 4

A solid fragrancing composition in stick form according to the invention was prepared as described below. The ingredients and the proportions thereof are indicated in Table 4 below.

TABLE 4

| | | | Fragrancing |
|---|---|---|---|
| | | Commercial | composition |
| Compounds | INCI name | reference | (g) |
| Carnauba wax | Copernica cerifera | Carnauba Wax | 6 |
| | (carnauba) wax/Copernica | 1 Flakes NF | |
| | cerifera cera | SP 63 | |
| Candelilla wax | Euphorbia cerifera | 7820 Light | 9 |
| | (candelilla) wax/Candelilla | Special | |
| | cera | Candelilla | |
| | | Real | |
| Hydrogenated jojoba | Hydrogenated jojoba oil | Jojoba Wax | 13 |
| oil | | Flakes | |
| Caprylic/capric acid | Caprylic/capric triglyceride | Miglyol 812 N | 18 |
| triglycerides | | | |
| Cetyl palmitate | Cetyl palmitate | Cutina CP | 3 |
| Castor oil | Ricinus communis (castor) | Crystal O | 15 |
| | seed oil/Ricinus communis | | |
| | seed oil | | |
| Refined sesame oil | Sesamum indicum (sesame) | Sesame Seed | 15 |
| | seed oil/Sesamum indicum | Oil Type C | |
| | seed oil | | |
| Dibutyl pentaerythrityl | Pentaerythrityl tetra-di-t- | Tinogard TT | 0.5 |
| tetrahydroxycinnamate | butyl | | |
| | hydroxyhydrocinnamate | | |
| Tocopherol | Tocopherol | Novatol 5-87 | 0.5 |
| | | 410218 | |
| Fragrancing substance | Natural perfume | | 20 |

All of the ingredients, except for the perfume, are mixed together and melted at 98° C., the perfume being introduced last so as not to deteriorate it, and the formulation is then poured into a mold at 40° C.

The shear hardness of this solid composition of Example 4 is 250 g.

It was found that, during the application of the composition in stick form, by exerting a normal force i.e. a force identical to the force exerted when applying a lipstick to lips, the wand does not crumble on the substrate.

The odor obtained after applying the solid composition of Example 4 to the substrate is very much the same as that of the fragrancing substance alone. The solid composition of Example 4 notably in the form of a stick allowed an easy and circumscribed application to the place where the solid composition of the invention was applied, contrary to the application of the fragrancing substance alone applied directly to the substrate which is difficult to control on the precise place to be fragranced. Moreover, it was found that the odor, after application of the solid composition of the invention, on the skin remains significantly more intense than that of the fragrancing substance alone, even after 4 hours.

### Example 5

A solid fragrancing composition in stick form according to the invention was prepared as described below. The ingredients and the proportions thereof are indicated in Table 5 below.

TABLE 5

| | | | Fragrancing |
|---|---|---|---|
| | | Commercial | composition |
| Compounds | INCI name | reference | (g) |
| Carnauba wax | Copernica cerifera | Carnauba Wax | 6 |
| | (carnauba) wax/Copernica | 1 Flakes NF | |
| | cerifera cera | SP 63 | |
| Candelilla wax | Euphorbia cerifera | 7820 Light | 9 |
| | (candelilla) wax/Candelilla | Special | |
| | cera | Candelilla | |
| | | Real | |
| Hydrogenated jojoba | Hydrogenated jojoba oil | Jojoba Wax | 13 |
| oil | | Flakes | |
| Caprylic/capric acid | Caprylic/capric triglyceride | Miglyol 812 N | 13 |
| triglycerides | | | |
| Cetyl palmitate | Cetyl palmitate | Cutina CP | 3 |
| Castor oil | Ricinus communis (castor) | Crystal O | 15 |
| | seed oil/Ricinus communis | | |
| | seed oil | | |
| Refined sesame oil | Sesamum indicum (sesame) | Sesame Seed | 15 |
| | seed oil/Sesamum indicum | Oil Type C | |
| | seed oil | | |
| Dibutyl pentaerythrityl | Pentaerythrityl tetra-di-t- | Tinogard TT | 0.5 |
| tetrahydroxycinnamate | butyl | | |
| | hydroxyhydrocinnamate | | |
| Tocopherol | Tocopherol | Novatol 5-87 | 0.5 |
| | | 410218 | |
| Fragrancing substance | Perfume | | 25 |

All of the ingredients, except for the perfume, are mixed together and melted at 98° C., the perfume being introduced last so as not to deteriorate it, and the formulation is then poured into a mold at 40° C.

The shear hardness of this solid composition of Example 5 is 130 g.

It was found that, during the application of the composition in stick form, by exerting a normal force i.e. a force identical to the force exerted when applying a lipstick to lips, the wand does not crumble on the substrate.

The odor obtained after applying the solid composition of Example 5 to the substrate is very much the same as that of the fragrancing substance alone. The solid composition of Example 5 notably in the form of a stick allowed an easy and circumscribed application to the place where the solid composition of the invention was applied, contrary to the application of the fragrancing substance alone applied directly to the substrate which is difficult to control on the precise place to be fragranced. Moreover, it was found that the odor, after application of the solid composition of the invention, on the skin remains significantly more intense than that of the fragrancing substance alone, even after 4 hours.

### Example 6

A solid fragrancing composition according to the invention in concrete form (in a dish) was prepared as described below. The ingredients and the proportions thereof are indicated in Table 6 below.

TABLE 6

| | | | Fragrancing |
|---|---|---|---|
| | | Commercial | composition |
| Compounds | INCI name | reference | (g) |
| Carnauba wax | Copernica cerifera | Carnauba Wax | 4 |
| | (carnauba) wax/Copernica | 1 Flakes NF | |
| | cerifera cera | SP 63 | |
| Candelilla wax | Euphorbia cerifera | 7820 Light | 8 |
| | (candelilla) wax/Candelilla | Special | |
| | cera | Candelilla | |
| | | Real | |
| Hydrogenated jojoba | Hydrogenated jojoba oil | Jojoba Wax | 11 |
| oil | | Flakes | |
| Caprylic/capric acid | Caprylic/capric triglyceride | Miglyol 812 N | 28 |
| triglycerides | | | |
| Cetyl palmitate | Cetyl palmitate | Cutina CP | 3 |
| Castor oil | Ricinus communis (castor) | Crystal O | 15 |
| | seed oil/Ricinus communis | | |
| | seed oil | | |
| Refined sesame oil | Sesamum indicum (sesame) | Sesame Seed | 15 |
| | seed oil/Sesamum indicum | Oil Type C | |
| | seed oil | | |
| Dibutyl pentaerythrityl | Pentaerythrityl tetra-di-t- | Tinogard TT | 0.5 |
| tetrahydroxycinnamate | butyl | | |
| | hydroxyhydrocinnamate | | |
| Tocopherol | Tocopherol | Novatol 5-87 | 0.5 |
| | | 410218 | |
| Fragrancing substance | Perfume | | 15 |

All of the ingredients, except for the perfume, are mixed together and melted at 98° C., the perfume being introduced last so as not to deteriorate it, and the formulation is then poured into a dish at 20° C.

The hardness of the solid fragrancing composition in the form of a concrete (in a dish) of Example 6 was assessed by measuring the shear hardness according to the abovementioned protocol for the same composition formulated in the form of a stick. The solid fragrancing composition in the form of a concrete of Example 6 has a shear hardness of the order of 130-140 g.

### Example 7

A solid fragrancing composition according to the invention in concrete form (in a dish) was prepared as described below. The ingredients and the proportions thereof are indicated in Table 7 below.

TABLE 7

| | | | Fragrancing |
|---|---|---|---|
| | | Commercial | composition |
| Compounds | INCI name | reference | (g) |
| Polyglycerolated | Polyglyceryl-3 beeswax | Cera Bellina | 4 |
| beeswax | | E00168 from | |
| | | Koster Keunen | |
| Candelilla wax | Euphorbia cerifera | 7820 Light Special | 5 |
| | (candelilla) | Candelilla Real | |
| | wax/Candelilla cera | | |
| Hydrogenated jojoba oil | Hydrogenated jojoba oil | Jojoba Wax Flakes | 5 |
| Caprylic/capric acid | Caprylic/capric | Miglyol 812 N | 32 |
| triglycerides | triglyceride | | |
| Cetyl palmitate | Cetyl palmitate | Cutina CP | 3 |
| Castor oil | Ricinus communis | Crystal O | 15 |
| | (castor) seed oil/Ricinus | | |
| | communis seed oil | | |
| Refined sesame oil | Sesamum indicum | Sesame Seed Oil | 15 |
| | (sesame) seed | Type C | |
| | oil/Sesamum indicum | | |
| | seed oil | | |
| Tocopherol | Tocopherol | Novatol 5-87 | 1 |
| | | 410218 | |
| Fragrancing substance | Perfume | | 25 |
| Kaolin | Kaolin | Imercare 04K | 5 |
| | | from Imerys | |

All of the ingredients, except for the perfume, are mixed together and melted at about 83-88° C., the perfume being introduced last so as not to deteriorate it, and the formulation is then poured into a dish at 20° C.

The hardness of the solid fragrancing composition in the form of a concrete (in a dish) of Example 7 was assessed by measuring the shear hardness according to the abovementioned protocol for the same composition formulated in the form of a stick. The solid fragrancing composition in the form of a concrete of Example 7 has a shear hardness of the order of 130-140 g.

### Example 8

A solid fragrancing composition according to the invention in concrete form (in a dish) was prepared as described below. The ingredients and the proportions thereof are indicated in Table 8 below.

TABLE 8

| | | | Fragrancing |
|---|---|---|---|
| | | Commercial | composition |
| Compounds | INCI name | reference | (g) |
| Polyglycerolated | Polyglyceryl-3 beeswax | Cera Bellina | 4 |
| beeswax | | E00168 from | |
| | | Koster Keunen | |
| Candelilla wax | Euphorbia cerifera | 7820 Light Special | 5 |
| | (candelilla) | Candelilla Real | |
| | wax/Candelilla cera | | |
| Hydrogenated jojoba | Hydrogenated jojoba oil | Jojoba Wax Flakes | 5 |
| oil | | | |
| Caprylic/capric acid | Caprylic/capric | Miglyol 812 N | 26 |
| triglycerides | triglyceride | | |
| Cetyl palmitate | Cetyl palmitate | Cutina CP | 3 |
| Stabilized | Butyrospermum parkii | Fair For Life | 3 |
| deodorized refined | (shea) butter/ | Refined Shea | |
| shea butter | Butyrospermum | Butter from Olvea | |
| | parkii butter | | |
| Stabilized shea olein | Butyrospermum parkii | Lipex 204 FFL | 3 |
| | (shea) butter/ | from | |
| | Butyrospermum | Aarhuskarlshamn | |
| | parkii butter | | |
| Castor oil | Ricinus communis | Crystal O | 15 |
| | (castor) seed oil/Ricinus | | |
| | communis seed oil | | |
| Refined sesame oil | Sesamum indicum | Sesame Seed Oil | 15 |
| | (sesame) seed | Type C | |
| | oil/Sesamum indicum | | |
| | seed oil | | |
| Tocopherol | Tocopherol | Novatol 5-87 | 1 |
| | | 410218 | |
| Fragrancing | Natural perfume | | 15 |
| substance | | | |
| Kaolin | Kaolin | Imercare 04K from | 5 |
| | | Imerys | |

All of the ingredients, except for the perfume, are mixed together and melted at about 83-88° C., the perfume being introduced last so as not to deteriorate it, and the formulation is then poured into a dish at 20° C.

5 The hardness of the solid fragrancing composition in the form of a concrete (in a dish) of Example 8 was assessed by measuring the shear hardness according to the abovementioned protocol for the same composition formulated in the form of a stick. The solid fragrancing composition in the form of a concrete of Example 8 has a shear hardness of the order of 130-140 g.

1. Solid fragrancing composition, notably a cosmetic composition, said composition being anhydrous and comprising:
i) at least 2% by weight, relative to the total weight of said fragrancing composition, of one or more fragrancing substance(s);
ii) one or more natural waxes and/or waxes of natural origin; and
iii) one or more natural fatty substances and/or fatty substances of natural origin, other than said natural wax(es) and/or wax(es) of natural origin ii).

2. Solid fragrancing composition according to claim 1, characterized in that it comprises at least 5% by weight, in particular at least 8% by weight, notably from 8% to 40% by weight, preferably from 9% to 30%, more preferentially from 10 to 25% by weight of fragrancing substance(s) i), relative to the total weight of said composition.

3. Solid fragrancing composition according to either of the preceding claims, characterized in that said fragrancing substance(s) i) are chosen from essential oils, perfumes and aromas of synthetic or natural origin, and mixtures thereof, preferably from natural fragrancing substances.

4. Solid fragrancing composition according to any one of the preceding claims, characterized in that the content of natural wax(es) and/or wax(es) of natural origin ii), preferably of ester wax type, is between 5% and 50% by weight, in particular between 8% and 40% by weight, more particularly between 10% and 35% by weight, notably between 15% and 30% by weight and preferably between 20% and 30% by weight, relative to the total weight of said composition.

5. Solid fragrancing composition according to any one of the preceding claims, characterized in that said natural wax(es) and/or waxe(s) of natural origin ii) are chosen from hydrocarbon-based waxes, in particular ester waxes and more particularly chosen from carnauba wax, beeswax, notably polyglycerolated beeswax, candelilla wax, hydrogenated jojoba oil and mixtures thereof;
preferably said composition comprises a mixture of natural waxes and/or waxes of natural origin comprising or even being formed from:
carnauba wax and/or beeswax, which is notably polyglycerolated, in particular in a content ranging from 1% to 15%, in particular from 2% to 10%; notably from 2% to 8% by weight and more particularly from 3% to 7% by weight, relative to the total weight of said fragrancing composition;
candelilla wax, in particular in a content ranging from 1% to 15% by weight, in particular from 2% to 12% by weight, more particularly from 4% to 12% by weight, notably from 5% to 10% by weight, relative to the total weight of said fragrancing composition; and
hydrogenated jojoba oil, in particular in a content ranging from 5% to 25% by weight, in particular from 8% to 20% by weight and more particularly from 10% to 15% by weight, relative to the total weight of said fragrancing composition.

6. Solid fragrancing composition according to any one of the preceding claims, characterized in that it comprises, as natural fatty substance(s) and/or fatty substance(s) of natural origin iii), other than said natural wax(es), at least one volatile or nonvolatile natural oil and/or volatile or nonvolatile oil of natural origin, preferably at least one oil of plant origin, in particular chosen from hydrocarbon-based oils of plant origin, notably from ester oils of plant origin; and more preferentially from:
oils of plant origin with a high triglyceride content, preferably chosen from castor oil, sesame oil, in particular refined sesame oil, caprylic/capric acid triglycerides, and mixtures thereof;
fatty acid monoesters of plant origin, in particular chosen from isopropyl myristate, myristyl myristate, isopropyl palmitate, cetyl palmitate, stearyl stearate and mixtures thereof, preferably cetyl palmitate;
and mixtures thereof.

7. Solid fragrancing composition according to the preceding claim, characterized in that it comprises a total content of natural oil(s) and/or oil(s) of natural origin, preferably ester oil(s) of plant origin, ranging from 20% to 80% by weight, in particular from 30% to 75% by weight and more particularly from 40% to 70% by weight, relative to the total weight of said composition.

8. Solid fragrancing composition according to any one of the preceding claims, characterized in that it comprises at least one solid natural fatty substance and/or solid fatty substance of natural origin, other than said natural wax(es), chosen from pasty fatty substances, notably butters, gums, and mixtures thereof.

9. Solid fragrancing composition according to any one of the preceding claims, characterized in that it comprises at least 40% by weight of natural fatty substances and/or fatty substances of natural origin, including at least one or more natural waxes ii), preferably between 40% and 98% by weight, in particular between 50% and 90% by weight, and more particularly between 59% and 80% by weight, of natural fatty substances and/or fatty substance of natural origin, including at least one or more natural waxes and/or waxes of natural origin ii), relative to the total weight of said fragrancing composition.

10. Solid fragrancing composition according to any one of the preceding claims, characterized in that it comprises:
at least 2% by weight, preferably at least 5% by weight, in particular at least 8% by weight, notably from 8% to 40% by weight, preferably from 9% to 30%, more preferentially from 10 to 25% by weight of fragrancing substance(s) i), relative to the total weight of the composition; preferably chosen from fragrancing substance(s) of natural origin;
carnauba wax and/or beeswax, which is notably polyglycerolated; in particular in a content ranging from 1% to 15%, in particular from 2% to 10%; notably from 2% to 8% by weight and more particularly from 3% to 7% by weight;
candelilla wax, in particular in a content ranging from 1% to 15% by weight, in particular from 2% to 12% by weight, more particularly from 4% to 12% by weight, notably from 5% to 10% by weight;
hydrogenated jojoba oil, in particular in a content ranging from 5% to 25% by weight, in particular from 8% to 20% by weight and more particularly from 10% to 15% by weight;
cetyl palmitate, in particular in a content ranging from 0.1% to 15% by weight, in particular from 0.5% to 10% by weight, notably from 1% to 8% by weight and more particularly from 1% to 5% by weight;
triglycerides, in particular in a content ranging from 5% to 80% by weight, in particular from 10% to 80% by weight, notably from 20% to 75% by weight and more particularly from 30% to 70% by weight, notably from 40% to 65% by weight; and
optionally one or more additives, in particular chosen from fillers, dyestuffs, cosmetic active agents, for example preserving agents, antioxidants, UV-screening agents and emollients; preferably one or more antioxidant compounds, for example chosen from tocopherol and/or dibutyl pentaerythrityl tetrahydroxycinnamate; preferably in a content ranging from 0.01% to 5.0% by weight, preferably from 0.05% to 2% by weight;
the percentages being given relative to the total weight of said solid fragrancing composition.

11. Solid fragrancing composition according to any one of the preceding claims, characterized in that it is in the form of a stick or is cast in a dish or jar, preferably in the form of a stick.

12. Cosmetic process for fragrancing keratin materials, in particular the skin, and notably the pulse points or the lips, comprising the application of a solid fragrancing composition as defined in any one of claims 1 to 11 to said keratin materials.
